(19) United States
(12) Patent Application Publication
(10) Pub. No.: US 2021/0065902 A1
(43) Pub. Date: Mar. 4, 2021

(54) Method And Apparatus For Planning A Procedure

(71) Applicant: Medtronic, Inc., Minneapolis, MN (US)

(72) Inventors: Andrew J. Cleland, Saint Paul, MN (US); Brooke G. KELLEY, Brooklyn Park, MN (US)

(21) Appl. No.: 16/998,411

(22) Filed: Aug. 20, 2020

Related U.S. Application Data
(60) Provisional application No. 62/892,266, filed on Aug. 27, 2019.
(60) Provisional application No. 62/892,287, filed on Aug. 27, 2019.

(51) Int. Cl. G16H 50/20; G16H 70/20; G16H 50/70; G16H 40/67; G16H 20/30

(52) U.S. Cl. CPC G16H 50/20; G16H 70/20; A61N 1/36071; G16H 40/67; G16H 20/30; G16H 50/70

(57) ABSTRACT

A system to assist in diagnosing and/or determining treatment for a selected subject. A user interface may be used to identify selected regions of a subject that have selected signs or symptoms. Guidance may be retrieved and/or updated based on inputs.

### CROSS-REFERENCE TO RELATED APPLICATIONS

This application claims the benefit of U.S. Provisional Application Nos. 62/892,266, filed on Aug. 27, 2019 and 62/892,287, filed on Aug. 27, 2019. The entire disclosures of all the above applications are incorporated herein by reference. This application includes subject matter similar to that disclosed in concurrently filed U.S. Patent Application No. __/___,___, (Attorney Docket No. 5074N-000034-US). The entire disclosures of all the above applications are incorporated herein by reference.

### FIELD

The subject application is directed to a system for therapy application, and particularly to a lead placement system and method for a therapy application.

### BACKGROUND

This section provides background information related to the present disclosure which is not necessarily prior art.

A subject, such as a human subject, may have treatment applied thereto for various concerns. For example, the subject may encounter or experience a discomfort or pain over a period of time, such as during movement. Various times, however, a subject may experience pain without any precipitating occurrence, such as not due to an injury or other initiating cause. The subject, therefore, may experience extended pain with no immediate treatment effect. In various instances, therefore, a treatment may include a stimulation to attempt to eliminate or overcome a pain or a concern felt by the subject. Various treatments may include implantable stimulators that excite or stimulate a portion of the subject's anatomy to treat or reduce perceived pain or discomfort.

### SUMMARY

This section provides a general summary of the disclosure, and is not a comprehensive disclosure of its full scope or all of its features.

A system may be used by a user, such as a medical practitioner including a surgeon, to assist in diagnosing and/or determining treatment for a selected subject. In various embodiments, a user interface may be used to identify selected regions of a subject that have selected signs or symptoms. The user interface may be used to identify and/or display information regarding possible or proposed therapy and perceived or possible outcomes. In various embodiments, the user interface may allow a user to operate a computer system to retrieve and/or update certain therapies and/or predicted outcomes of therapies.

A user interface may include a touch sensitive device and/or a display that allows a user to interact with the display. The display may display information regarding a subject, possible diagnostic techniques, and other information for selection by a user. The user may select the various diagnostic signs and symptoms and the display may further display various possible outcomes based upon selected treatments. The signs and symptoms may be predefined for assisting in diagnosing a selected syndrome or disease, such as displaying and/or allowing selection of the Budapest Criteria to assist in diagnosing a selected issue, such as complex regional pain syndrome (CRPS).

A selected system may collect information regarding various therapies and results of therapies. A follow up procedure may allow a selected user to follow up with a subject after a procedure is performed to determine the success of a therapy based upon specifics of the therapy. The follow up diagnostic criteria and information may be used to determine or alter proposed therapies based upon predetermined criteria. The user interface may allow for accessing the proposed criteria for introducing or suggesting therapies to a patient in the future.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a schematic representation of a procedure room;

FIG. 2 is a screen shot of a clinical device, according to various embodiments;

FIG. 3 is a screen shot of the clinical device illustrating possible pain locations;

FIG. 4 is a screen shot of the clinical device showing possible location of a pain;

FIG. 5 is a screen shot of the clinical device illustrating a heat map;

FIG. 6 is a screen shot of the clinical device illustrating a possible heat map, according to various embodiments;

FIG. 7 is a screen shot of the clinical device illustrating implement for patient history;

FIG. 8 is a screen shot of the clinical device illustrating possible device programming guidance;

FIG. 9 is a screen shot of the clinical device illustrating follow up information;

FIG. 10 is a screen shot of the clinical device illustrating an image of a subject;

FIG. 11 is a flow chart of a guidance determination process; and

FIG. 12 is a detailed view of clinical guidance method.

Corresponding reference numerals indicate corresponding parts throughout the several views of the drawings.

### DETAILED DESCRIPTION

Example embodiments will now be described more fully with reference to the accompanying drawings.

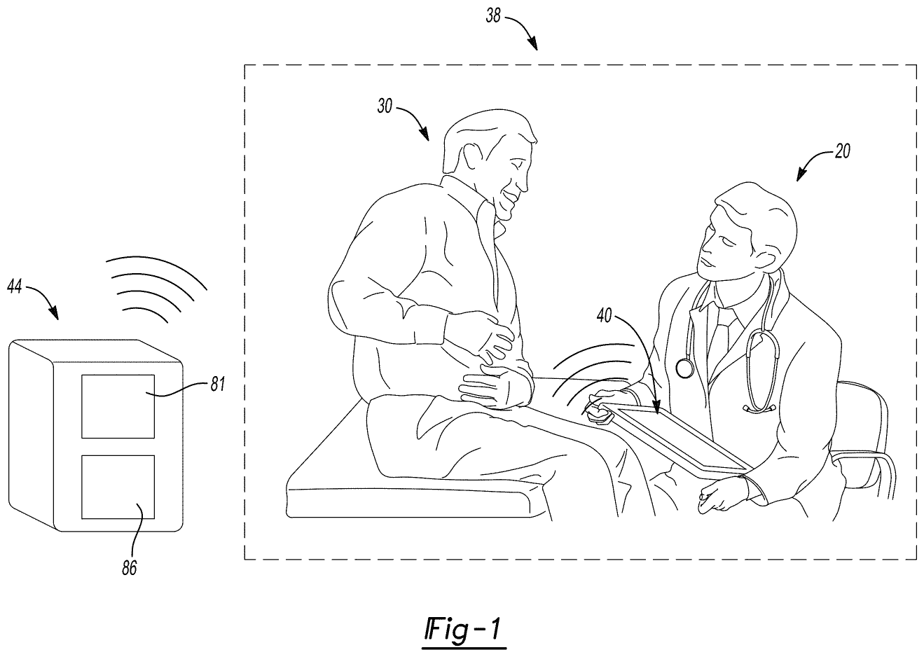

With initial reference to FIG. 1, a user 20 may meet a subject 30 in a selected setting, such as a clinical setting. The user 20 may use a planning system or diagnosis guidance system 38. In various embodiments, a clinical device 40 may be used by the user 20. The clinical device 40 may also be reference to herein as the system 40 and may include one or more inputs (e.g. touchscreen) and/or a display 50.

The user may use or implement the selected system 40 to assist with operating the system 38. The system 40 may include a single device such as a tablet or other processor or computing device. The system 40 may be operable to be portable and housed for use by the user 20.

The system 40 may be used by the user 20 to access selected information and/or input information regarding the subject 30 in the guidance system 38. The system 40 may, therefore, be a personal or clinical device and may communicate with an external device or database device 44 which may be away from the clinical setting. The database or external system 44 may include various features or portions such as a processor 81 and a memory system 86. The processor 81 may access and/or recall information from the memory 86. Further, the device 40 may include, separate from the external device 44, an internal processor and/or memory to access and/or execute instructions from the internal memory.

The clinical device 40 may be in communication by a communication system with the external device 44, such as via wireless communication, wired communication, or other appropriate data transfer mechanisms. It is understood, however, that the external device 44 may be incorporated into the system 40 and that external communication is not necessary. Regardless, the clinical device 40 may be used by the user 30 to access information regarding the specific patient 30, access general information regarding selected diagnosis and/or proposed treatments, and/or retrieve or determine relevant guidance regarding proposed treatment and/or in light of a diagnosis.

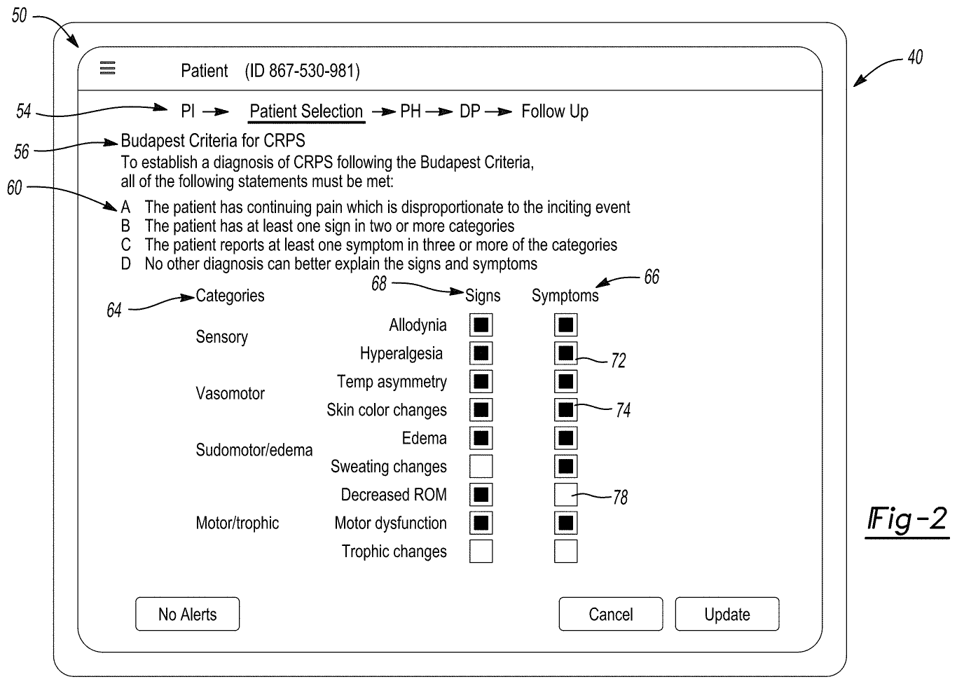

With continuing reference to FIG. 1, and additional reference to FIG. 2, a display 50 may be displayed on the clinical device 40 for use and input by the user 20. The display 50 may be any appropriate display, such as a touch sensitive display, to act as an input device. It is understood that other appropriate input devices may be used for user input with an input device such as a keyboard or a mouse, or other appropriate mechanism. Nevertheless, the display 50 may be displayed for viewing by the user 20. The device 40, such as through or with the display 50, may be used by the user 20 to select or identify various features or categories, as discussed herein, of the subject 30 and/or select the subject 30. Accordingly, a first screen may include inputting or selecting patient information that may be selected in a main menu or menu bar 54. The main menu 54 may include various selections that may be operated or selected by the user 20. Accordingly, the user 20 may select the patient info or information menu to select or enter information regarding a patient.

Once patient information is included, patient diagnosis and/or selection may occur by selecting a menu item such as patient selection. The user 20 may also select a one or more selected criterion and/or determination, such as diagnosis for CRPS and/or from selected categories to assist in a diagnosis. The user 20, using the device 40, may access selected criterion, such as the Budapest Criteria, to assist in making or confirming a diagnosis of CRPS. The user 20 may then input with the device 20 selected criteria for a diagnosis technique, such as a Budapest Criteria 56.

Displayed with the device 40 may be various criteria for determination of a selected diagnosis, such as for CRPS. The criteria may include the standard criteria or inherent criteria 60. The selected criteria or inherent criteria may include appropriate information for selected diagnosis. For example, to diagnosis CRPS all or a selected number of the Budapest Criteria may include all or certain criteria must be met, such as subject continuing pain with a disproportionate inciting event, (e.g. movement without impact), patient has at least one sign of two or more categories, patient reports at least one symptom in three or more categories, and no other diagnosis explains the signs and symptoms. In various embodiments or diagnosis, signs may include specific criteria that may be felt and/or identified by a user, such as the user 20. Symptoms may be self-reported issues in the categories. Accordingly, the patient 30 may identify symptoms by self-reporting and the user 20 may identify signs of the subject 30. A user selection may include specific or user based criteria in addition to and/or alternative to selected or predetermined criteria (e.g. the Budapest criteria).

During use, the user 20 may review categories, such as the categories 64. The user 20 may then put into the system with the device 40 symptoms selected from a symptom column 66 and signs selected from a sign column 68. The user 20 may select the categories and identify the sign or symptom relative thereto, such as with a check box or fillable criteria item 72. A filled or completed box 74 may be identified with a check or other demarcation, such as being changed in color from an unfilled box 78. Accordingly, the user 20 may identify the signs or symptoms for the subject 30 and input them with the device 40. The information input of a device 40 may be used to access a database of selected information and therapies, such as by accessing the storage system 44, which may also be an external or offsite system. Thus, the input in the system 40 may be transmitted to the external device 44. It is understood, however, that the external device may be incorporated into the system 40 such that input (including the signs and symptoms and user preferences) may be used by the external or guidance system 44, as discussed herein, regardless of an external transmission or not. An external transmission from the system 40 to the external system 44 is, however, possible.

The offsite system 44 may include selected memories, such as random access or other memory types that may be accessed with commands from the device 40. The offsite system 44 may also include the processor 81, to receive instructions from the device 40 by input of the user 20 and recall and transmit information from the storage 86. Accordingly, the processor 81 may receive the command from the device 40, such as through input of the user 20, and access the memory 86. The accessed memory may then be processed with the processor 81 and/or transmitted for display on the device 40, as discussed further herein.

Generally, categories for the Budapest Criteria may include sensory categories such as allodynia which includes pain or sensitivity to light, touch, pressure, or the like or hyperalgesia to a pin prick. Other categories include vasomotor, which includes temperature asymmetry and skin color changes or asymmetry, such as noting or identifying temperature or skin color changes on the subject 30. Other categories include sudomotor or edema, such as sweating changes and/or asymmetry or fluid collection in the subject. Finally, motor/trophic changes may include a decrease in range of motion, motor dysfunction (such as weakness or tremors) and/or trophic changes (such as changes in hair, nail, skin, or other dogmatic changes). The user 20 may input the identified signs and symptoms 68, 66 with the device 40 and may view the inputs on the display 50. The inputs may include these diagnostic inputs (e.g. the signs and symptoms) and other appropriate inputs. Generally, however, the diagnostic inputs may also include a patient history, if selected. The diagnostic inputs may be used by the system 38 to provide one or more guidance sets to the user 20 via the system 40, as discussed herein.

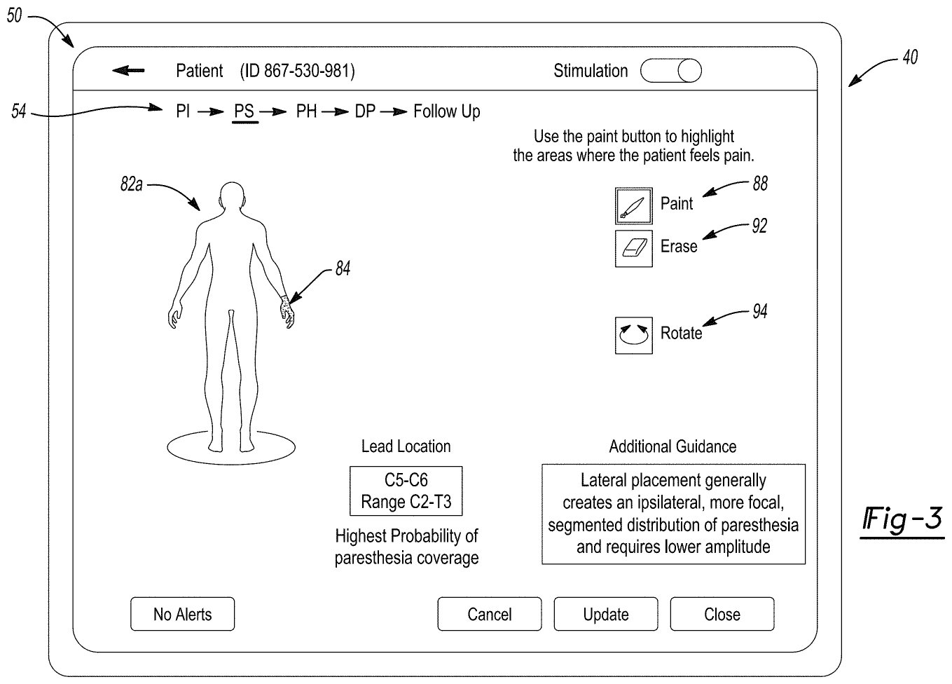

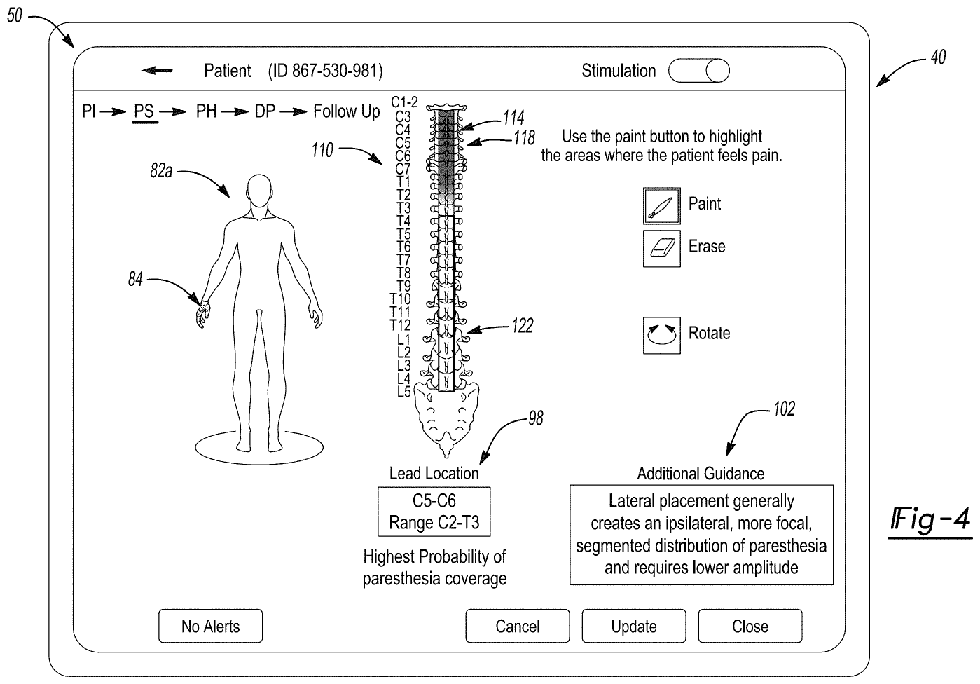

With continuing reference to FIG. 1 and additional reference to FIG. 3 and FIG. 4, the device 40 may be used by the user 20 to input information regarding pain location on the subject 30. The input may include or be referred to as diagnostic input. As discussed herein, the diagnostic input may include patient selection or characteristics and may be transmitted for selected guidance, as discussed herein.

Inputs may include, for example, with reference to FIG. 3, the display 50 may display a graphical representation 82 of the subject 30. The graphical representation may be any appropriate representation such as a graphical display (also referred to as an icon), a photograph of the subject 30, or other appropriate displays. The user 20 may then use the device 40 to input pain locations. By inputting a pain location, the user 20 may use the graphic 82 as a map and, therefore, map the pain location. It is understood that other appropriate individuals may also use the device 40 to input pain locations, such as the subject 30, or other observer. Regardless, input of pain locations may be made with the device 40.

For example, the user 30 may touch a selected portion of the display 50, in the instance of a touch sensitive display. Selected touch sensitive displays may include those generally known in the art including capacitive, pressure, and a like sensitive displays. Regardless, the graphical representation 82 of the subject 30 may then be altered to display the selected pain location as a pain map. For example, a color change or marking of a hand 84 may be made to illustrate that a right hand of a subject has been indicated as a pain location. It is understood that any other appropriate portion of the subject may also be selected. The user 20 may select to indicate the selection or mapping by activating a selection or paint icon 88. The user 20 may also change or unselect portions by selecting an erase or removal icon. It is understood that any appropriate tools may be used, such as using a hardware device selection, software device selection, or the like. Regardless, the user 20 may identify and select various portions of the graphical representation 82 to identify pain locations.

With continuing reference to FIG. 3 and additional reference to FIG. 4, the pain location may be at various portions of the subject 30, such as on an anterior or posterior portion. Accordingly, a rotation or change orientation icon 94 may be used to cause the graphical representation 82 of the subject 30 to be rotated or moved. As illustrated in FIG. 4, an anterior view of the subject may be displayed and additional or other locations of pain may be identified.

An anterior graphical representation may be identified as anterior graphic 82a. It is understood that the display 50 may display on a single screen the graphical representation 82 of the subject 30 and the single graphical representation may be rotated, zoomed, magnified, or otherwise altered for viewing by the user 20. Accordingly, the user 20 may view the graphical representation 82 and rotate the graphical representation 82 while continuing to view the other portions of the display 50, such as the various activation tools or icons 88, 92, 94. Thus, the user 20 may also mark alternative or additional areas that may only be on an anterior or a posterior portion of the subject. Nevertheless, the graphical representation 82a may also illustrate the color change or pain location indication 84 such as by a color change of the right hand of the subject graphical representation 82a.

Once the user 20 has identified all pain locations, or even while pain locations are being identified, the display 50 may also display various guidance or suggestions based upon analysis of the input criteria. Guidance is exemplary illustrated in FIG. 2 based on pain locations identified in FIG. 3 and FIG. 4 and recalled information from the external system 44. Guidance information may be provided on the same display 50 with the graphical representation 82 of the subject and/or on a separate display. The planning system 38 may include a plurality of selected guidance sets (e.g. including one or more guidance information) and a selected or relevant guidance may be displayed based on the inputs and analysis thereof.

Guidance information may be displayed at various portions of the display 50. The display 50 may change and alter based upon recalled information from the external system 44 and/or analyzing inputs from the user 20 such as pain locations and criteria designations. The inputs may be diagnostic inputs (e.g. regarding the subject 30). The guidance displayed may be relevant guidance based on the analysis of the inputs by the system 38. The guidance may include a plurality of portions (e.g. lead type and placement), thus the guidance may be a guidance set. Further, the displayed guidance may be one determined from a plurality of guidance sets.

Various guidance sets may each include selected guidance or elements of guidance. The set may include discrete guidance such as a possible lead location or locations for positioning or implanting stimulation leads in a lead location guidance box 98. Additional guidance or additional lead placement guidance may be provided in block 102 and may include a specific placement of a lead relative to a selected placement of a lead within a subject. For example, the additional guidance box 102 may indicate that lateral placement and/or type of lead that may be positioned within the subject 30. For example, various stimulation leads may include a plurality of nodes or focal points that may be operated individually or sequentially and additional guidance may provide output based upon pain location and other criteria, as discussed further herein.

Additionally, guidance sets may include information such as a heat map or display 110. The heat map display 110 may display various portions of the anatomy of the subject 30, such as a spinal column including an illustration of a plurality of the vertebrae 114. The vertebrae may include all of the vertebrae of the subject or those vertebrae for which guidance is recommended or appropriate. For example, for pain location in the right hand 84, cervical vertebrae and/or vertebrae in the cervical and superior thoracic region may be identified in a guidance set. Accordingly, the heat map 110 may include only those vertebrae or may include all vertebrae in a display, such as a graphical representation of the spine. Regardless, the heat map 110 may further include a graphical illustration, such as a color bar or gradation, including a first color bar or gradation 118. The gradation or color bar may include a color change and/or color density change to identify the range of vertebrae recommended or guidance for placement based upon inputs. The heat map 110 may include a secondary color map 1222 that may be formed in a separate color or gradation (e.g. striped as opposed to dotted) to identify a second heat map that may include portions that are not within the relevant guidance set or not recommended for the inputs thus far. Regardless, the heat map 110 may include guidance regarding positioning of a lead placement for treatment of the subject 30 based upon inputs such as pain location and diagnosis criteria. Further, the heat map and/or other guidance may be updated as inputs are received, such as in real time.

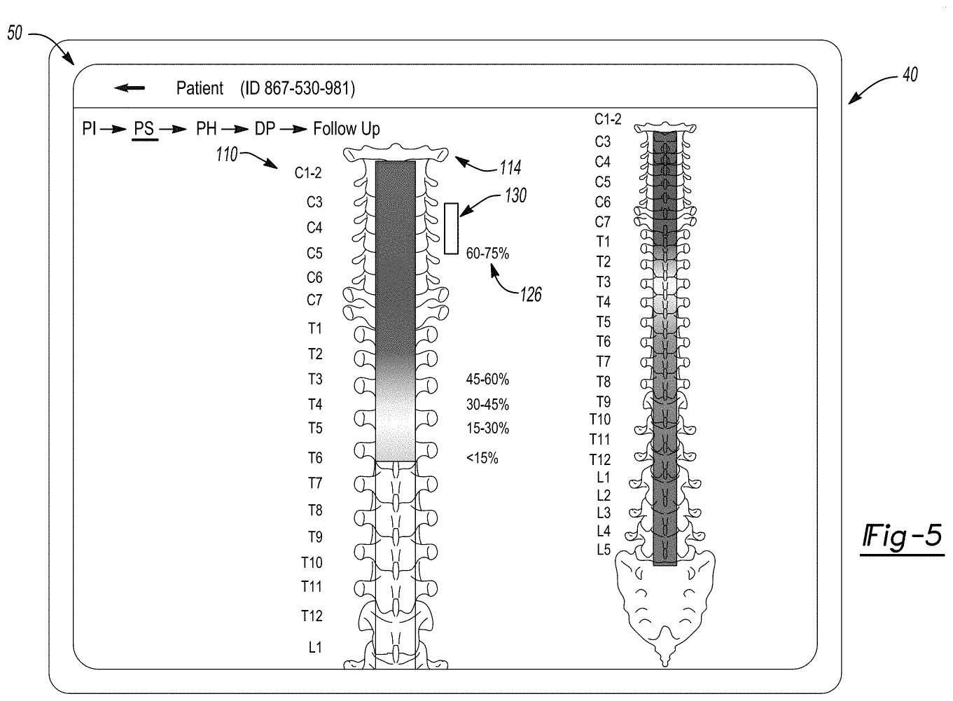

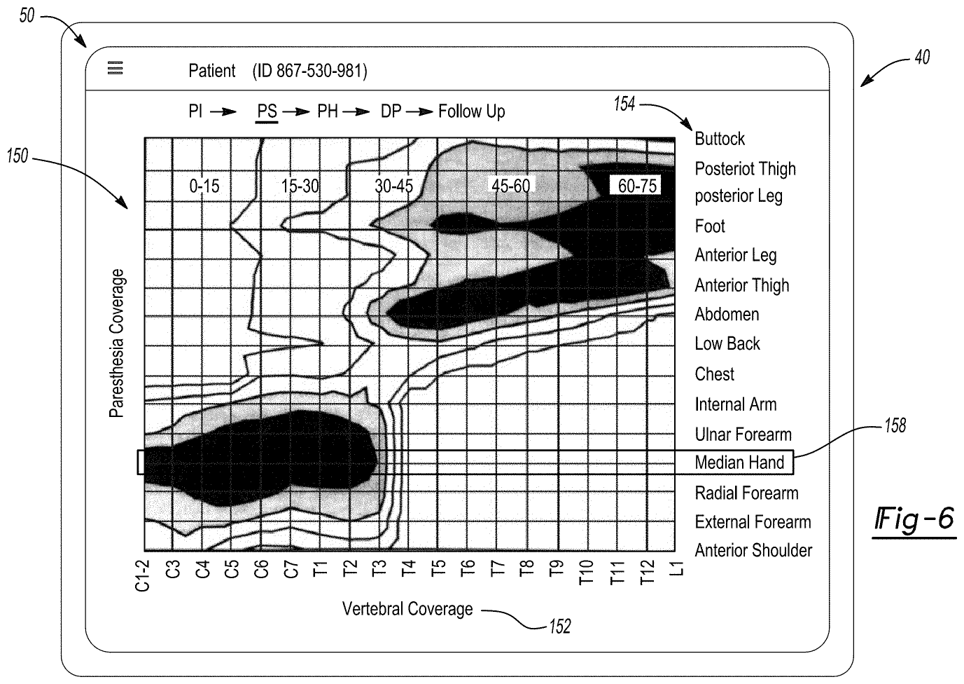

With continuing reference to FIGS. 3 and 4 and additional reference to FIGS. 5 and 6, the heat map 110 may be displayed on a separate screen display or screen shot of the screen 50. Accordingly, based upon input, the user 20 may view the heat map 110 separate from the graphical representation 82 of the subject 30. The heat map 110 may be substantially identical to the heat map 110 illustrated in FIG. 4, but may include additional information such as success rates, such as a percentage 126 for a selected location, as a part of the guidance set.

The heat map 110 may also be altered or changed by the user 20 such as by zooming or changing orientation. For example, the user 20 may move a graphic illustration or icon 130 that may represent a lead implant. The success rate 126 may be altered based upon the proposed position of the lead by the user 20 moving the lead icon 130. Accordingly, the user 20 may move the lead icon relative to the heat map 110 including a representation of the spinal column 114. The success rate may then be updated based upon information recalled from the external device 44, as discussed further herein. The user 20 may then also view the guidance on the display 50, as illustrated in FIG. 4, regarding suggested placements and/or types of leads.

With reference to FIG. 6, the user 20 may select to view alternative and/or additional heat maps, such as an alternative heat map 150. The alternative heat map 150 may be based upon additional or alternative information that may be recalled from the external system 44. The selection or display of the heat map 150 may be based upon an input from the user 20, such as signing in and using the device 40. For example, a specific user account may recall for display a particular type of heat map. The heat map 150 may illustrate a representation of a vertebra, such as on a vertebra level 152. The heat map 152 may also provide information regarding a specific location of pain along a y-axis 154. The guidance may include a demarcation or highlighting 158 of a specific or guided location based upon selected information, as discussed further herein.

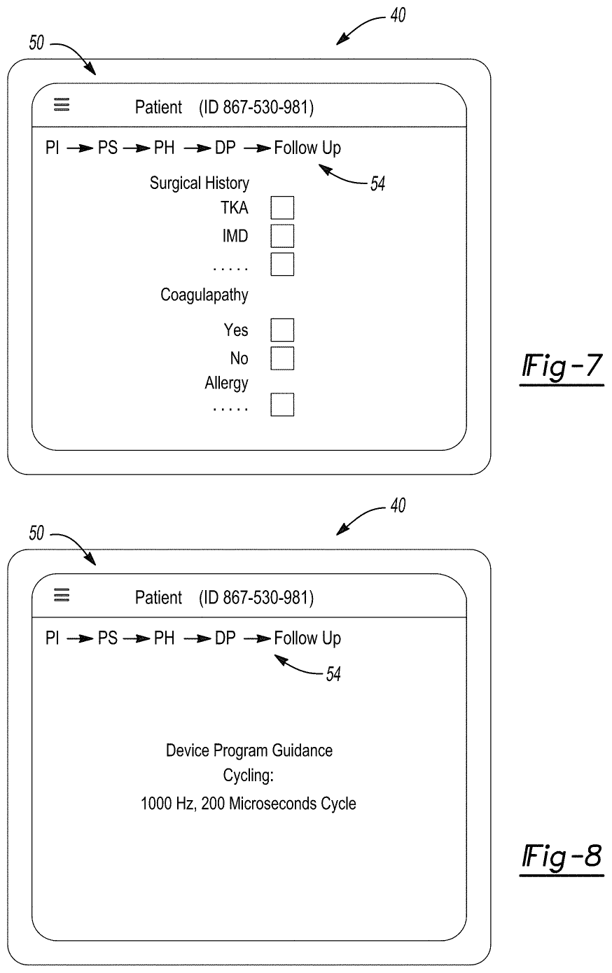

With reference to FIG. 7, the user 20 may include additional information regarding the subject or patient 30, such as including a patient history. The patient history may include various information such as surgical history, coagulopathy, allergies, or the like. A surgical history may include a determination or selection of whether selected surgical procedures have been performed in the past, such as spinal fusion and/or location, a total knee arthroplasty, implantable medical device (e.g. pacemaker) or other surgical procedures. An indication of whether coagulopathy is present or diagnosed in the subject may also be made. For example, a type of procedure to occur on the subject may be altered or placement of a lead may be selected depending upon a type of procedure that occurs or additional steps that maybe occur during an implant procedure. Further, allergy information may be entered. Allergy information may be relevant for a length of procedure, type of implant, or the like. Accordingly, the user 20 may input information regarding a patient's history to again assist in determining a type of therapy.

Turning reference to FIG. 8, the guidance may include guidance regarding device programming (DP) including programming of an implantable device, such as the stimulation device. Accordingly, device programs may be displayed on the display 54 to the user 20 with the device 40. As discussed above, determining relevant guidance may include accessing the external device 44 with the system 38, as discussed further herein, to assist in identifying guidance for a selected treatment based upon the determined input from the user 20.

The displayed or retrieved guidance may include, for example, illustrating a cycling program. The program may include a simulation at 1000 hertz cycled every 200 microseconds. The programming may also include duration of treatment such as cycling 200 microseconds at 1000 hertz and 200 microseconds at 0 hertz or no stimulation, and then cycling again at the stimulation frequency. Therefore, the programming guidance may be provided to the user 20 for selected purposes. As discussed further herein the selected guidance or provided guidance may be based upon selected information and determinations as discussed herein.

Accordingly, the system 38, including the device 40, may be used to provide to the user 20 a relevant guidance set for a selected procedure based on inputs from the user 20 for treatment for the patient 30. The relevant guidance may be based on analysis, as discussed herein, regarding a plurality of guidance sets. The user 20 may receive or evaluate information regarding the patient 30 and make a determination for treatment thereof based upon the diagnosis of the subject 30. The relevant guidance may also be augmented by preference of the user 20 (e.g. selecting lateral placement).

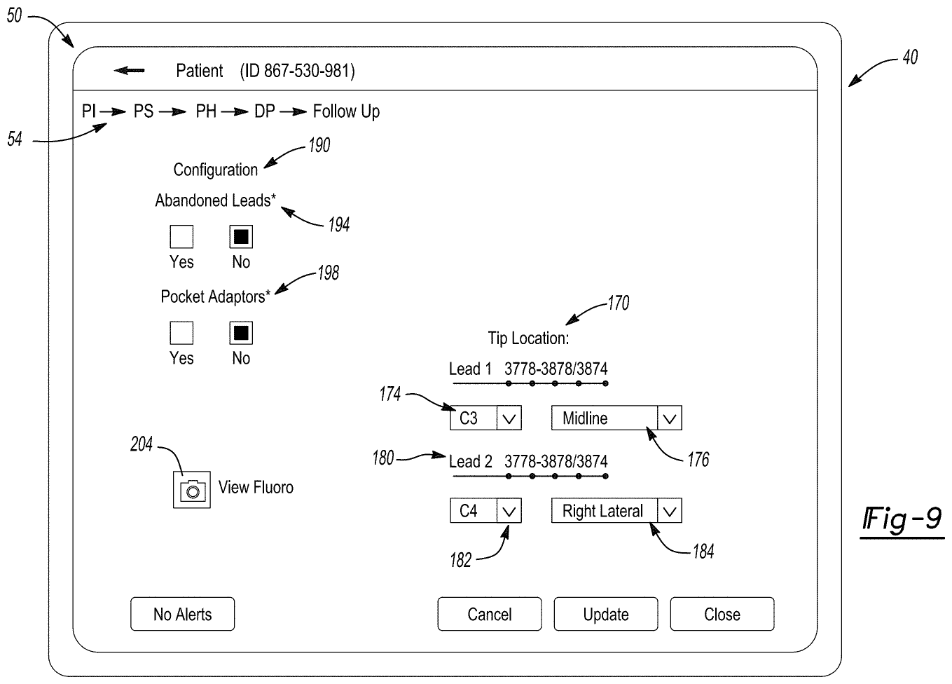

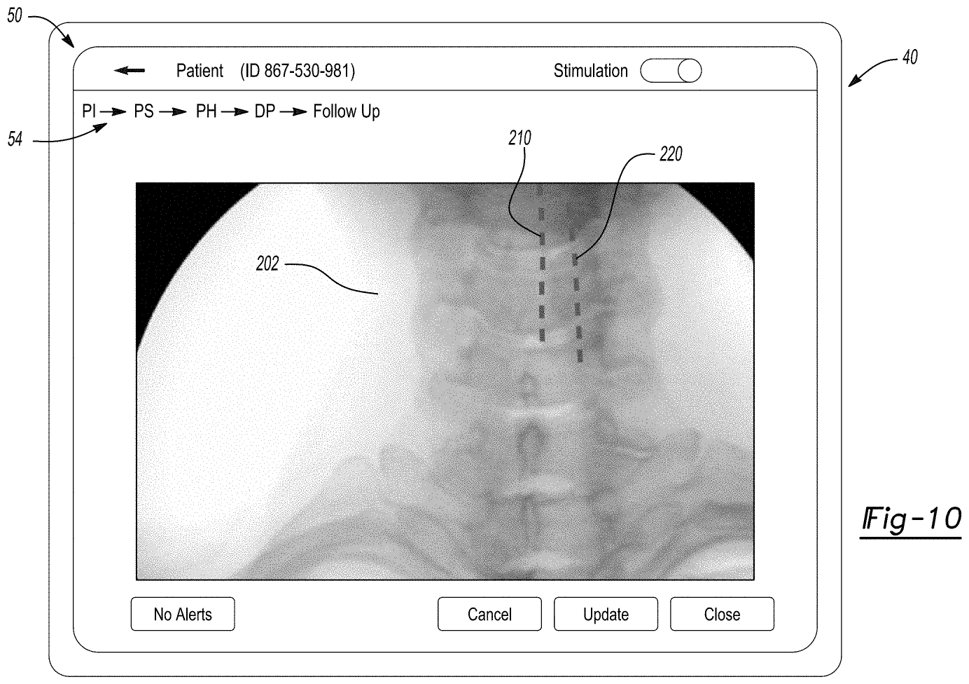

The system 38, including the device 40, may also be used for various follow up or secondary procedures. For example, with reference to FIG. 9 and FIG. 10, the user 20 may input into the system 38, such as with the device 40 to transfer to the external device 44, information regarding the subject 30. Follow up information or inputs may be collected after positioning an implant and/or during a selected follow up period. For example, the user 20 may implant a device based upon a selected determination, which may or may not follow exactly the relevant guidance provided or discussed above. Nevertheless, during a selected follow-up period, including immediately after implantation, or a selected follow-up time thereafter, the user 20 may collect information regarding the subject and/or placement of leads in the subject.

For example, as illustrated in FIG. 9, the user 20 may identify a lead type and/or location per data entry portion 170. The user may determine or identify a lead implant location (e.g. at cervical vertebrae 3) 174 and an amount of laterality, such as at midline 176. If more than one lead is used, the user 20 may identify the second lead at block 180. Additionally, the location may be entered 182 and the amount of laterality 184 may also be entered for the second lead identified at 180.

Also, the user 20 may include other additional information for the subject including lead configuration information 190. Configuration may include whether other leads have been abandoned, such as when entering a yes or no, or selecting a mark box 194. Other information, such as whether a pocket adaptor is used at block 198, may also be input. It is understood that other information may be provided by the user 20 and may be entered into the system 38, including the internal system 44, for further use, as discussed herein. Further, specific information provided by the user 20 may also be added and/or provided as an additional data field that may be used by a selection or guidance system.

Further, image information may be provided, such as fluoroscopic images 202 that may be viewed by selecting the recall button 204. The images may be stored in selected imaging systems and/or the external system 44. The fluoroscopic image 202 may illustrate the position leads, such as two leads including a first lead 210 and a second lead 220. The position of the two leads may be illustrated in the image 202, which may include a direct image of the subject 30 after positioning of the leads. The image, therefore, may be used for later analysis including determination of a specific placement of the lead, such as with a specific position relative to midline (e.g. such as a distance). The fluoroscopic image 202 may be also used to assist in follow up, such as for comparison to later acquired images (e.g. after the acquisition of the image 202) and for other purposes.

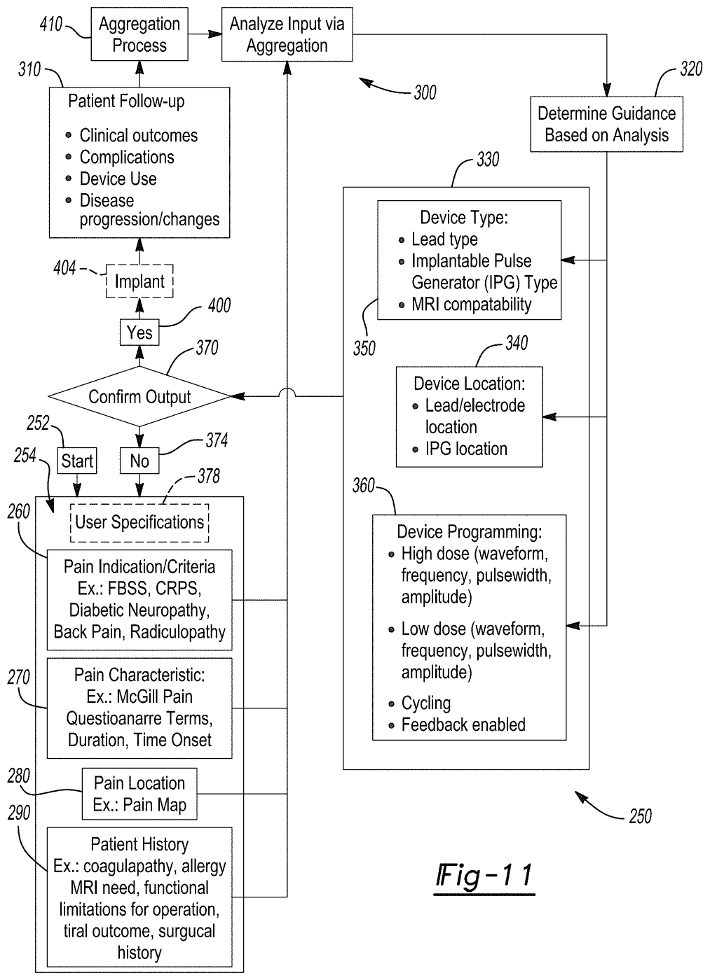

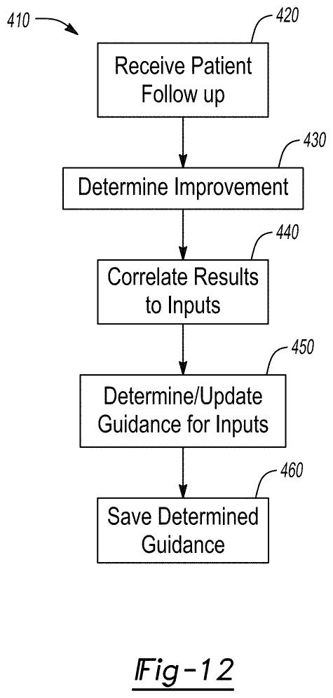

Accordingly, the user 20 may use the device 40, or any other appropriate device, to input information regarding the subject 30. The external system 44 may be used as a part of a guidance system 38 to assist in providing guidance. Guidance or a guidance set may be based upon analysis or use of a guidance selection process 250, as illustrated in FIG. 11 and FIG. 12.

With reference to FIG. 11 and FIG. 12, the method 250 may include various features and steps, as discussed herein. These may be implemented in various manners, such as by the user 20 inputting with the device 40 in the planning and guidance system 38 and/or communicating (e.g. sending or receiving data) with the external system 44. For example, the user 40 may input various information with the device 40. Inputs may include those in input block 254. The system 38, however, may also be incorporated into a single device such as the device 40. Further, as discussed herein, an aggregation process 410 may receive inputs from a plurality of users 20 and/or subjects 30 and be accumulated over time.

The inputs may be inputted with the user interface, such as including the graphical user interface 50, as discussed above. The inputs 254 may include various information and may also be referred to as and/or part of diagnostic inputs. The inputs 254 may include a pain indication or criteria inputs 260. The inputs may include the CRPS criterion, back pain (e.g. location, type, frequency), diabetic neuropathy pain, radiculopathy, or other back pain syndromes, such as failed back pain surgery symptom (FBSS). As noted above, for example CRPS, certain criteria or signs and symptoms may be incorporated into a selected diagnosis for specific type of pain or diagnosis. Accordingly, the pain indication may include a general pain indication selection screen (e.g. back pain) with specific criteria based upon different possible diagnosis based upon the criteria. As discussed above and illustrated in FIG. 2, a diagnosis screen or patient selection screen may include information regarding specific existence of signs or symptoms for selected categories. Thus, the user 20 may input specific information regarding the pain of the subject 30 to assist in selection for treatment guidance.

Further inputs 254 may include pain characteristics in block 270. Pain characteristics may include various types of pain, such as a stabbing or throbbing pain, burning pain, or others. Examples include the McGill pain questionnaire terms. Other pain characteristics may include duration, initiation time, etc. Accordingly, the user 20 may indicate, such as input with the device 40, the types of pain experienced by the patient 30. As discussed above, the signs or symptoms may include the pain characteristic which may be entered at block 270.

Pain location may be entered, as discussed above. In various embodiments, such as with a pain map to identify the location of the pain such as with the representation 82, discussed above. The user 20 and/or the subject 30 may identify a location of pain by indicating on the graphical representation 82 location of the pain. It is further understood that the location of pain may be input in other manners such as identifying a specific location (e.g. right hand) from a list, or entering a specific location. Regardless, the pain location may be entered in block 280.

Further, a patient history may be entered in block 290. The patient history may include patient history, as noted above, including various allergies, MRI needs, functional limitations for or during an operation, trial outcomes, prior surgical history, coagulopathy, or other patient history. The patient history again may be provided for selection by the user 20 with the screen 50. Specific or relevant patient history criteria may be presented and the user 20 may select and identify the relevant history for the subject 30. The user 20, therefore, may input to the system 38 relevant history. The method 250 may include a list of relevant histories or type of history for a selected patient and/or selected types of pains or pain diagnosis. Thus, the history may be limited to or specific to a specific subject and/or type of pain. Regardless, the user 20 may identify or select the patient history and input the same in block 290.

Following the input 254, the method 250 may receive or analyze the inputs with a current guidance model and/or aggregation information in block 300. The aggregation information may include guidance regarding selected treatments for various inputs, such as pain indications or criteria, pain characteristics, pain location, etc. The aggregation information may include information regarding specific follow up and/or general follow up information collected from prior procedures that have been analyzed or aggregated from a patient follow up 310, as discussed further herein.

The aggregation data and/or database may include a set or range of relevant guidance for various features or inputs, as discussed further herein. Accordingly, analyzing the input may include recalling from a database specific or ranges of various outputs, as discussed further herein, for treating the subject 30 based upon the inputs 254.

After analyzing the input via the aggregation, a determination of a determined or relevant guidance may be based upon the analysis in block 320. The determination of guidance may include the analysis of the inputs. The analysis may include recall from a database based upon follow up and prior results of procedures that correlate to the inputs 254. The determined guidance may be based upon the specific inputs from the user 20. Further, the relevant guidance may include and/or be limited to specific selections and/or desires of a particular user. For example, although a determined guidance may include positioning of a lead at a selected level (e.g. C4), a specific user may include placement of the lead laterally from a midline based upon a specific user's preference or selection. Accordingly, the determined guidance in block 320 may include user specific guidance. It is understood that other appropriate guidance may be included, such as may be selected by the user 20.

Regardless, the determined guidance from block 320 may be output as relevant guidance in block 330. The output guidance may be displayed on the device 40, as discussed above. The displayed guidance may include indicating a heat map position for placement of a lead in a spinal column, indication of the lateral placement, or other information in block 340. The device or lead location may include lead or electrode location. It is understood by one skilled in the art, a lead may include a plurality of electrodes, such as a tip electrode and a proximally displaced electrode and/or a plurality of proximally displaced electrodes. Accordingly, guidance may include an indication of a lead including one or more electrodes and/or positioning of electrodes. Additionally the device location in block 340 may include implantable pulse generator (IPG) location guidance. As discussed above, the device location guidance may be displayed on the screen 50 of the device 40 after the inputs 254 made by the user 20 or other appropriate individual.

Relevant guidance may include device type, as noted above. For example, device type may include a lead type such including one or more electrodes. Device type may also include IPG type. Further device type guidance may include MRI compatibility and/or type. The device type may include a specific type of delivery or implantable device in addition to the specific lead for positioning within the subject 30. Accordingly, the device type guidance in block 350 may include device guidance that may be positioned or implanted in the subject 30 for treatment of the pain input in block 254.

Additionally, the output guidance 330 may include device programming in block 360. Device programming in block 360 may include various guidance, such as determining whether a low or high dose should be provided. A low or high dose may include a frequency and/or amplitude of a treatment. For example a low dose may be treatment at about 200 hertz and a high dose may be a treatment at about 1,000 hertz. It is understood that the device programming, however, may include a range of dose and/or a specific guidance dose amount. Further, the device programming guidance may include cycling, such as duration of the dosing and/or duration between dosing times. Cycling may be provided as guidance for programming the implantable device. Device programming may include allowing for feedback enabled or controlling of the dosage to the subject 30. Accordingly, the specific programming of the device may include receiving feedback from the user and allowing for augmentation of the specific dosing and/or cycling based thereon.

The output 330 may be displayed for the user 20 on the display 50 of the device 40. It is understood that the output may be displayed in any appropriate display for the user 20, such as an alternative display device, print out, transmission to a supply house, or the like. Regardless, the user 20 may review the guidance displayed with the device 40.

The user 20 may, optionally, also determine whether the output is appropriate such as through confirmation in block 370. The user 20 may view the output 330 and confirm or determine that alteration of the output may be selected and/or desired. If the output is not confirmed in block 370, a NO path 374 may be followed to the input block 254. The user 20 may then alter or change inputs in block 254. Additionally, the user 20 may include a specific additional input, such as a specific selected lead, in a user specifications block 378. The user specifications 378 may be an optional input in block 254. Particularly, if the output is not confirmed in block 370 and the NO-path 374 is followed, the user specification input block 378 may be enabled in the system 38 to allow for the user to include user specifications. The user specification 378, as noted above, may include various specifics such as a specific lead and/or specific type of dose. The inputs 254 may then be reanalyzed in block 300 to determine additional or different guidance in block 320 and output in block 330. The output may then again be confirmed in block 370 repeatedly until output is confirmed and a YES-path 400 is followed. Thus, the user 20 may select specific treatment in addition to or alternative to the relevant guidance of the output 390.

Once a YES-path is followed 400, a procedure may follow to implant the selected device type at the selected device location and the device may then be programmed in block 404. The implantation is optional as a part of the method 250, as patient follow up may occur in block 310 regardless of the specific implantation.

The patient follow up in block 310 may allow for a collection of data regarding the patient or subject 30 after the procedure, such as the implantation in block 404. The patient follow up 310 may include collection or entering specific information regarding the placement of the lead and device and the programming thereof, as noted above and may include images of the subject 30 regarding placement of leads. The patient follow up 310 may also include or collect information regarding clinical outcomes for the subject 30 including a disease progression or change. The user 20, or any appropriate user, may collect data from the subject 30 including a signs and symptoms similar to the inputs 254.

The follow up data collected 310 may be from a plurality of subjects 30 and/or procedures. Selected data may also be collected from procedures not used within the system 38. Regardless of the source, follow up data (e.g. results of selected procedures) may be aggregated. The aggregated data with the specific placement and programming of the device for aggregation, as discussed further herein. Follow up of the subject 30 may include determining or recording any complications following the implantation and device usage. For example, the subject 30 and/or user 20 may allow for intermittent use of the device after implantation and the amount or use or continued use of the device may be monitored and recorded. Regardless of the patient follow up information collected in block 310, the follow up information may be included in an aggregation method or algorithm 410.

The patient follow up which includes all of the information regarding whether the output 330 was followed and/or precise information regarding the device type, location, and program and the results thereof. The aggregation process 410, therefore, includes the information regarding the specifics of the input 254 and the specifics of the actual treatment (e.g. device type, device location, and device programming), and the results or follow up 310 thereof. The aggregation process 410, therefore, may be used to generate an aggregation database or recall guidance that is analyzed with the input or is used to analyze the input in block 300.

With continuing reference to FIG. 11 and additional reference to FIG. 12, the aggregation process 410 is illustrated in greater detail. The aggregation process 410 may include receiving patient follow-up in block 420. Receiving patient follow-up may include receiving to the external system 44 the patient follow up collected in block 310. The patient follow up 310 may include all the information as discussed above, including patient diagnosis and treatment and results of the treatment.

A determination of improvements may then be made in block 430. A determination of improvements may include a treatment that resulted in an improvement of a selected criteria, such as a pain indication or pain characteristic following a specific treatment compared to a current or selected guidance set. For example, for CRPS with pain in a right hand after treatment with a lead of a specific programming at C4 lead or resulted in patient follow up with improved results a determination of improvement may occur. Particularly, the determination may include comparing results of a plurality of subjects. For example, a first subject with a set of inputs 254 that may be substantially identical or are identical to inputs for a second subject where a particular treatment led to an improvement (e.g. reduced pain characteristics), then a specific type of treatment may be indicated as an improvement. Accordingly, the improvement may be determined in block 430.

Once the determination of improvements is made in block 430, a correlation of results to inputs may be made in block 440. For example, inputs of a specific type or specific set of inputs may be correlated to patient follow up regarding the specific set of inputs. A plurality of subjects or a plurality of different subjects with substantially identical inputs from which patient follow up information is input or collected, a correlation between all of the identical inputs and the patient follow up may be made. Accordingly, the treatments may be ranked based upon a patient follow up results and correlated to the specific inputs to allow for a correlation of treatments including outputs 330 that correlates to improved feedback or follow up in block 310.

Once the correlation of the results to inputs is made in block 440, a determination or update of guidance for inputs is made in block 450. As discussed above, a first set of guidance may be updated if a treatment output resulted in more patient treatment to result in improved patient follow up. Accordingly, guidance for placement of a lead at C4 for a specific set of inputs 254 may be altered if an actual treatment that received improved patient feedback or follow up is made, such as for lead placement at C3 rather than C4. Accordingly, the guidance may be update or determined in block 450. The updated guidance may include information from the follow-up that led to improved results for the subject.

The guidance may then be saved in block 460. The saved guidance in block 460 may be saved as a database for recall by the external system 42 and/or device 40. Generally the saved guidance may be accessed in block 300 for determination of guidance in block 320.

Accordingly, as discussed above, the user 20 may input the inputs 254, such as with the device 40. Based on the inputs 254, an analysis of the inputs may be based on the aggregation data 410 to determine guidance based on the analysis to revive guidance outputs 330. The guidance outputs 330 may be displayed on the display 50 for review by the user 20. The user 20 may confirm the output in block 370, as discussed above. Thus, the user 20 may operate the system 38, such as using the device 40, to assist in diagnosing the subject 30 and/or determining treatment or determining treatment guidance for the subject 30.

Relevant guidance, therefore, may be displayed with the device 40 within the guidance system 38. The relevant guidance may be based on diagnostic inputs from the user 20 (or other appropriate input) within analysis based on follow up and other later collected information. The relevant guidance any assist or provide guidance for a selected treatment.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

Instructions may be executed by a processor and may include may include software, firmware, and/or microcode, and may refer to programs, routines, functions, classes, data structures, and/or objects. The term shared processor circuit encompasses a single processor circuit that executes some or all code from multiple modules. The term group processor circuit encompasses a processor circuit that, in combination with additional processor circuits, executes some or all code from one or more modules. References to multiple processor circuits encompass multiple processor circuits on discrete dies, multiple processor circuits on a single die, multiple cores of a single processor circuit, multiple threads of a single processor circuit, or a combination of the above. The term shared memory circuit encompasses a single memory circuit that stores some or all code from multiple modules. The term group memory circuit encompasses a memory circuit that, in combination with additional memories, stores some or all code from one or more modules.

The apparatuses and methods described in this application may be partially or fully implemented by a special purpose computer created by configuring a general purpose computer to execute one or more particular functions embodied in computer programs. The computer programs include processor-executable instructions that are stored on at least one non-transitory, tangible computer-readable medium. The computer programs may also include or rely on stored data. The computer programs may include a basic input/output system (BIOS) that interacts with hardware of the special purpose computer, device drivers that interact with particular devices of the special purpose computer, one or more operating systems, user applications, background services and applications, etc.

The computer programs may include: (i) assembly code; (ii) object code generated from source code by a compiler; (iii) source code for execution by an interpreter; (iv) source code for compilation and execution by a just-in-time compiler, (v) descriptive text for parsing, such as HTML (hypertext markup language) or XML (extensible markup language), etc. As examples only, source code may be written in C, C++, C #, Objective-C, Haskell, Go, SQL, Lisp, Java®, ASP, Perl, Javascript®, HTML5, Ada, ASP (active server pages), Perl, Scala, Erlang, Ruby, Flash®, Visual Basic®, Lua, or Python®.

Communications may include wireless communications described in the present disclosure can be conducted in full or partial compliance with IEEE standard 802.11-2012, IEEE standard 802.16-2009, and/or IEEE standard 802.20-2008. In various implementations, IEEE 802.11-2012 may be supplemented by draft IEEE standard 802.11ac, draft IEEE standard 802.11ad, and/or draft IEEE standard 802.11ah.

A processor or module or ‘controller’ may be replaced with the term ‘circuit.’ The term ‘module’ may refer to, be part of, or include: an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor circuit (shared, dedicated, or group) that executes code; a memory circuit (shared, dedicated, or group) that stores code executed by the processor circuit; other suitable hardware components that provide the described functionality; or a combination of some or all of the above, such as in a system-on-chip.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A system to plan and select treatment for a subject by a user, comprising:
a memory system having stored thereon a plurality of guidance sets;
a communication system configured to communicate with the memory system;
a display device configured to display a relevant guidance set selected from the plurality of guidance sets; and
a processor operable to execute instructions to:
analyze an inputted selected criterion,
determine the relevant guidance set from the plurality of guidance sets based at least on the analyzed inputted selected criterion, and
retrieve the relevant guidance set from the memory.

2. The system of claim 1, further comprising:
an input system configured to allow the user to input selected criterion; and
a first device comprising at least the input system, the communication system, and the display device.

3. The system of claim 2, wherein the memory system and the processor are operable to communicate with the first device via the communication system;
wherein the first device is operable to be remote from the memory system and the processor.

4. The system of claim 2, wherein the first device is operable to be held by the user in a single housing.

5. The system of claim 1, wherein the relevant guidance set includes at least two relevant guidance sets.

6. The system of claim 1, wherein the relevant guidance set includes a heat map of possible implantation sites of a stimulation lead.

7. The system of claim 6, wherein the user input is operable to configure the display device to display the relevant guidance in at least one of a plurality of configurations.

8. A method to plan and select treatment for a subject by a user, comprising:
communicating by a communication system with a memory system having stored thereon a plurality of guidance sets;
operating a processor to execute instructions to analyze a selected criterion and determine a relevant guidance set from the plurality of guidance sets based at least on the analyzed inputted selected criterion, and
operate a display device to display the relevant guidance set of the plurality of guidance sets.

9. The method of claim 8, further comprising:
inputting with an input system a selected criterion; and
operating the processor to retrieve the relevant guidance set from the memory.

10. The method of claim 9, further comprising:
inputting at least one user specific criterion; and
formatting the display of the relevant guidance based at least on the at least one user specific criterion to a user specific relevant guidance.

11. The method of claim 10, wherein the user specific relevant guidance includes displaying at least one of a specific lead type, a lead placement, a lead placement procedure, or combinations thereof.

12. The method of claim 10, wherein the user specific relevant guidance includes displaying a limited heat map of possible implantation sites of a stimulation lead.

13. The method of claim 8, further comprising:
inputting at least one user specific criterion;
recalling subject categories limited by the at least one user specific criterion; and
displaying the limited subject categories for viewing by the user.

14. The method of claim 8, wherein operating the processor to execute instructions to analyze the selected criterion and determine the relevant guidance set from the plurality of guidance sets comprises comparing the selected criterion to prior subject follow-up and correlated treatment.

15. A system to plan and select treatment for a subject by a user, comprising:
an input system configured to allow the user to input selected criterion;
a memory system having stored thereon a plurality of guidance sets;
a communication system configured to communicate with the memory system;
a display device configured to display a relevant guidance set from the plurality of guidance sets; and
a processor operable to execute instructions to:
analyze the selected criterion,
determine the relevant guidance set from the plurality of guidance sets, and
retrieve the relevant guidance set from the memory.

16. The system of claim 15, further comprising:
a clinical device comprising at least the input system, the memory system, the communication system, the display device, and the processor.

17. The system of claim 16, wherein the display device is configurable based on the input from the input system to determine a display image.
